(19) United States
(12) Patent Application Publication
(10) Pub. No.: US 2008/0220026 A1
(43) Pub. Date: Sep. 11, 2008

(54) Mesoporous material compositions and methods of their use for improving the appearance of biological surfaces

(76) Inventors: Prithwiraj Maitra, Randolph, NJ (US); John C. Brahms, Morris Plains, NJ (US)

Correspondence Address: Charles J. Zeller, Esq.;Avon Products Inc., Avon Place, Suffern, NY, 10901, US

(21) Appl. No.: 11/714,516

(22) Filed: Mar. 6, 2007

(51) Int. Cl. A61K 33/30; A61K 33/24; A61K 9/00; A61Q 19/08; A61Q 17/04; A61K 33/26

(52) U.S. Cl. 424/400; 424/617; 424/642; 424/646

(57) ABSTRACT

The present invention relates to compositions and methods for treating and improving the aesthetic appearance of a biological surface by altering the optical properties of the biological surface, thereby resulting in a natural appearance. The compositions of the invention may be topically applied to the biological surface, including but not limited to skin, in an amount effective in improving the aesthetic and natural appearance of the biological surface.

### FIELD OF THE INVENTION

This invention generally relates to cosmetic, dermatological, and pharmaceutical compositions and their use, and more particularly to cosmetic compositions and their use in improving the appearance of biological surfaces.

### BACKGROUND OF THE INVENTION

There is an increasing demand in the cosmetics industry to develop products that may be applied topically to the skin to improve the appearance of skin such that the skin has a natural appearance. Ideally, a biological surface, such as skin, would have a translucent appearance with uniform coloring, a smooth and even surface, with no apparent imperfections. Consumers are interested in mitigating or delaying the dermatological signs of chronologically- or hormonally-aged or photo-aged skin, such as fine lines, wrinkles, drying, and sagging skin, and other conditions due to a progressive degradation of the skin matrix, as well as improving the appearance of skin damaged by environmental factors, including sun, pollutants, smoking, etc. Therefore, there is a need for cosmetics that assist in creating a flawless, natural look. Aging skin is characterized by poor elasticity and the accentuation of lines and wrinkles due to reduced oil gland activity. Collagen, a fundamental protein in the composition of skin, plays a primary role in elasticity and hydration. As the aging process occurs, the collagen structure weakens, and skin loses its firmness becoming ineffective in retaining moisture. The breakdown of collagen and its precursors contributes to wrinkling and creates the appearance of sagging skin. Though reversing the aging process is not possible, the appearance of fine lines and wrinkles can be diminished. Topical application of products which make fine lines and wrinkles appear more smooth and plump are contemplated.

Achieving a perfect, flawless and natural look is difficult because cosmetic ingredients which provide the desired color and coverage, are generally opaque, thereby obscuring the vibrant and natural translucency of the skin. Pigments or particles with less opacity are available, but they do not provide enough coverage to mask or camouflage the flaws in or on the biological surface, including skin. Cosmetic ingredients can settle in the fine lines and wrinkles, accentuating the imperfections on the skin's surface. Therefore, there remains a need for cosmetic formulations and preparations that convey the perception that the consumer's skin has fewer wrinkles and imperfections by allowing the optimal refraction and reflection of light and increasing the diffusion of light to accomplish the foregoing.

Mesoporous materials have been found to be useful in optical and electronic functional materials (U.S. Pat. No. 5,958,577; “the '577 patent”). In particular, the '577 patent reports a method for producing a bulk silica porous material having a large crystal size, which reduces light scattering, and a uniform and adjustable pore size for optical and electronic functional materials.

There is a need for a composition and method of use thereof to improve the aesthetic appearance of skin. Therefore, safe and effective compositions that provide a translucent appearance in a film with uniform coloring, smooth and even biological surfaces, with no apparent imperfections would be advantageous for the formulation of treatments and products for biological surfaces, including the skin.

### SUMMARY OF THE INVENTION

It is an object of the present invention to provide a composition that delivers in an acceptable vehicle or carrier, an effective amount of a mesoporous material sufficient to alter the appearance of a biological surface, where the composition enhances diffused transmittance.

In another object of the invention, the mesoporous material of the composition is a metal oxide such as TiO₂, ZnO, Al₂O₃, a mesoporous silica, and combinations thereof,

A further object of the invention is to provide a composition having a mesoporous material with empty pores or loaded with material, including for example, metal nanocrystals, such as noble metal nanocrystals, and fluorescent materials, which further produce unique optical effects on skin.

In yet another object, a method of improving the aesthetic or natural appearance of a biological surface by applying to the biological surface a composition having a mesoporous material and carrier in an amount effective to improve the aesthetic or natural appearance of the biological surface. The composition is applied such that the appearance of dermatological signs of damaged or chronologically-aged, hormonally-aged, actinically-aged, photo-aged skin, such as fine lines, wrinkles, and sagging skin, surface imperfections, and discoloration is diminished.

Another object provides a method for forming a film on a biological surface, such as but not limited to a keratinous surface by contacting the surface with a mesoporous material and carrier composition, thereby forming a film on the keratinous surface, where the film produces blurring effect to reduce both color and textural imperfections and to improve the natural appearance of the keratinous surfaces. Mesoporous materials can produce these effects by achieving a balance between diffused transmittance and opacity and thus retaining natural translucency of biological tissues. Thus, reducing the appearance of lines, wrinkles, creases, crow's feet, suborbital lines, periorbital lines, hyperpigmentation, skin discoloration, blemishes, sagging skin, skin fragility, and/or skin atrophy is an object of the instant invention.

These and other objects and advantages of the present invention, and equivalents thereof, are achieved by compositions having a mesoporous material and combinations thereof, and methods of using such compositions for topical application in order to improve the aesthetic and natural appearance of a biological surface, including for example, skin.

### BRIEF DESCRIPTION OF THE DRAWING

FIG. 1 shows the effects of percent solid loadings with respect to percent diffused transmittance or percent reflectance of mesoporous TiO₂, compared to that of pigment grade. TiO₂.

FIG. 2 shows the effects of film thickness with respect to percent diffused transmittance of mesoporous TiO₂, compared to that of pigment grade TiO₂, at a 2.5% solid loading.

### DETAILED DESCRIPTION OF THE INVENTION

In accordance with the foregoing objectives and others detailed herein, the invention overcomes deficiencies associated with the prior art by providing compositions comprising at least one mesoporous material, and an acceptable vehicle, which improves the aesthetic appearance of biological surface resulting from, for example, the chronological aging process, environmental damage to the biological surface, or other imperfections. The composition and methods thereof, once applied to a biological surface or synthetic biological surface, enhances the appearance of the surface by altering the appearance of a biological surface by enhancing diffused transmittance and reflective properties, thereby improving the aesthetic appearance of the biological surface. The aesthetic appearance of the biological surface, including but not limited to, keratinous tissues, skin, hair, and nails, may be achieved by topical application of the inventive composition to the biological surface on a daily basis or when a natural appearance is desired.

In one embodiment of the invention, the composition embraces a mesoporous material having several properties that enhance diffused transmittance and results in a film with high blurring effect upon application of a thin film of the mesoporous material composition to a biological surface. The film is about 0.2 microns to about 50 microns in thickness; and preferably about 1 micron to about 20 microns.

Mesoporous materials may be of an ordered or disordered structure of pores, providing transparent and more preferably translucent properties when formed in a film. The pores may be loaded with another material in order to enhance the optical properties of the mesoporous material by altering the resulting refractive index. The pores may also be loaded, or simply empty but for air, or loaded with air. The mesoporous material and the material that is loaded in the pores of the mesoporous material are capable of altering the scattering behavior of visible light, thereby providing soft focus or blurring effects useful in cosmetic compositions which camouflage wrinkles, fine lines, or imperfections of biological surfaces.

Any mesoporous material of the inventive composition producing the desired effects may be used, non-limiting examples of which include a metal oxide, for example, titanium oxide (TiO₂) such as AEROXIDE® TiO₂ P25 (Degussa Corporation; Parsippany, N.J.) aluminum oxide, and iron oxide, mesoporous silica, or combinations thereof.

In yet another embodiment of the invention, the mesoporous material may be a hybrid, for example, a metal oxide and polymer. Combinations, hybrids or mixtures of a metal oxide and a polymer may be used as the film forming components of the composition described herein. Non-limiting examples of polymers include homopolymers and copolymers of: (1) synthetic polymers including vinyl esters that are produced by the polymerization of vinyl ethers (e.g., polyvinyl acetate, produced by free-radical initiated polymerization of vinyl acetate); (2) acrylic acid esters and methacrylic acid esters, readily combine with themselves or other monomers (e.g., amides, acrylonitrile, vinyl, styrene, and butadiene) by reacting at their double bond, forming homopolymers or copolymers which are useful in the invention; acrylic acid esters and methacrylic acid esters include, but not limited to, methyl acrylate, ethyl acrylate, butyl acrylate, 2-ethyhexyl acrylate, methyl methacrylate, ethyl methacrylate and butyl methacrylate; and (3) mono and di-ethylene unsaturated hydrocarbons, such as ethylene iso-butylene, styrene and aliphatic dunes, such as butadiene, isoprene and chloroprene.

In one embodiment, polyhedral oligosilsesquioxane is an example of hybrid mesoporous material. The polyhedral oligosilsesquioxane has randomly linked arrays of polycyclic cages of bridged polysilsesquioxanes by means of sol-gel polymerization may be used as well. These network polymers may be porous xerogels and aerogels, or non-porous polymers depending on whether the organic spacer is a rigid arylene group or a flexible alkylene group. Monolithic gels containing organic dyes have been prepared for waveguide, NLO (Non-linear optical), and laser applications. The dye function in these materials is built into the organic bridging group avoiding phase separation, aggregation and loss by leaching.

As an example, mesoporous TiO₂ has a large fraction of pores, about 50-80%, empty or filled with trapped air. As a result mesoporous TiO₂ has greater light diffusing properties compared to non-porous materials. Light scattering is reduced and the film comprising mesoporous materials appears more translucent than that of the non-porous materials. This film produces a blurred effect, reducing both color and textural imperfections and improving the natural appearance of the keratinous surfaces. Mesoporous materials can produce these effects by achieving a balance between diffused transmittance and reflectance and opacity, and thus retaining natural translucency of biological tissues. High reflectance provides optimal coverage, thereby obscuring discoloration and hyperpigmentation. These qualities combined result in a composition which may reduce the appearance of lines, wrinkles, creases, crow's feet, suborbital lines, periorbital lines, hyperpigmentation, skin discoloration, blemishes, sagging skin, skin fragility, skin atrophy and/or the like. These attributes are extremely important in achieving a blurring effect but also to improve the natural appearance of skin with good coverage. The balance between reflectance and diffused transmittance is essential for obtaining a mesoporous material which provides good coverage with optical blurring resulting in a natural appearance. A mesoporous material having a balance of these properties is optimal. For example, a film of the mesoporous material has a reflectance of less than about 55% providing sufficient coverage and a diffused transmittance of greater than about 18% providing optical blurring enables a good balance as determined by the method described in Example 1. The method uses a film that contains 5% mesoporous material and has a thickness of 10 microns. Other mesoporous material equivalents are also contemplated should they have the desired characteristics described herein.

In one embodiment of the invention, the mesoporous material has a high pore density and volume. These qualities in combination with the refractive index enable the composition to produce a blurring effect by enhancing diffused transmittance and thereby improving the appearance of the biological surface onto which the composition was applied. Without wishing to be bound by theory, the efficiency of the blurring and soft focus effect or the ability to camouflage fine lines and wrinkles is believed to be a function of the diffused transmittance parameter (“Quantification of the Soft Focus Effect”, Cosmetics & Toiletries, 111:57, 1996). Diffused transmittance is known as the portion of light that is scattered or diffuse by irregularities. Haze and clarity characteristics are both a measure of the degree of scatter and in combination make up diffused transmittance. While haze is a measure of wide-angle scattering, which causes a loss of contrast, clarity is the measure of narrow-angle scattering. Clarity and details are lost when an object is viewed through another material, such as a film, or when the object is further away. Accordingly, the higher the haze, the lower the clarity, and thus more diffusion occurs. Therefore, any mesoporous material that produces high diffused transmittance would be beneficial in a cosmetic formulation or preparation for producing a blurring effect, improving the natural appearance of the biological surface to which the cosmetic is applied, and overcoming the drawbacks of typical cosmetics that are opaque, unnatural and chalky, and those that settle in fine lines and wrinkles.

Having a high pore density and pore volume facilitates the blurring effect, by increasing diffused transmittance. The pore volume is about 0.1 cm³/g to about 2 cm³/g; more preferably about 0.1 cm³/g to about 1.8 cm³/g; and most preferably about 0.2 cm³/g to about 1.6 cm³/g, while the refractive index of the mesoporous material is about 1.38 to about 3.52. More preferably, the refractive index is about 1.40 to about 3.50; and most preferably about 1.42 to about 3.40. Because the refractive index of human skin is about 1.55, a preferred refractive index range of around the skin refractive index is useful for modifying the optical properties of skin. The refractive indices of various materials may be calculated using a refractometer. (See, for example, hypertext transfer protocol://world wide web2.ups.edu/faculty/hanson/labtechniques/refractometry/interpret.htm) Details with respect to refractive indices of materials may be found in the “CRC Handbook of Chemistry and Physics,” 86th Edition, 2005-2006, which is herein incorporated by reference in its entirety. The diameter of a pore is about 2 nanometers to about 70 nanometers; and preferably about 5 nanometers to about 60 nanometers. In another embodiment, the mesoporous material has a pore size such that the wavelength of the light that enters the pore has a wavelength less than that of visible light. A mesoporous material having a surface area in the range of 20 m²/g to 700 m²/g and preferably about 35 m²/g to about 500 m²/g is most useful in the inventive composition for producing a blurring and soft focus effect.

The advantages of the inventive composition include, but are not limited to, the simplicity of at least one mesoporous material to provide optical blurring and “coverage” without the drawbacks of pigments or dyes, such as pigmented TiO₂, which produces a chalky white residue thereby losing the soft focus effects and inadvertently accentuating the fine lines or wrinkles into which pigments settle. Non-mesoporous materials, including traditional commercial soft focus materials such as PMMA, PE, and spherical silica, provide soft blurring effects. However, they have a low refractive index, which result in a sheer film thereby providing little or no coverage.

In yet another embodiment of the invention, nanocrystals or nanoparticles may be loaded in the ordered or disordered pores of the mesoporous material. The term “nanoparticles” as used herein, can also refer to nanoclusters, clusters, particles, dots, quantum dots, small particles, nanostructured materials. These nanocrystals may be either placed or formed in situ in the pores of the mesoporous material using electrical and/or van der Waals interactions. Depending on the size of active ingredients typically used in cosmetic formulations, they should not be trapped in the mesoporous material pores because these pores are very small. However, should these pores be loaded with materials other than air, then these materials would alter the optical properties of the compositions.

One embodiment of the invention relates to mesporous material pores loaded with (noble) metal nanocrystals, including but not limited to, precious metals, gold, silver, copper, platinum, palladium, tantalum, and iridium. The optical spectra of noble metal clusters are known to demonstrate apparent resonance lines resulting from collective excitations of electrons. These electron excitations are known as particle plasmons, Mie plasmons or surface plasmons. A further embodiment loads any metal that is resistant to corrosion or oxidation and results in the absorption of light at specific wave lengths through the Mie plasmon effect thereby producing non-linear optical properties. The mesoporous materials loaded with, for example, (noble) metal nanocrystals, exhibit plasmon resonance effects resulting from non-linear optical properties which, would be useful as an alternative to cosmetic colorants, pigments, or dyes.

Fluorescent materials are known to produce visible or invisible light as a result of incident light of a shorter wavelength (i.e. X-rays, UV-rays, etc.). The effect ceases as soon as the source of excitement is removed. Fluorescent materials are also contemplated as one of the materials to load in the pores of the mesoporous material of the inventive composition. Non-limiting examples of fluorescent materials include zinc sulfide, Fluorescein isothiocyanate (FITC), and fluorescein derivatives, such as Oregon Green, Tokyo Green, SNAFL, and carboxynapthofluorescein.

If the mesoporous material comprises particles that are extremely small, for example, less than or about 100 nanometers, then the films and coatings may be produced that also provide ultraviolet (UV) protection. Another advantage of the mesoporous material is that it absorbs oil, sebum, and moisture, for example. These properties provide additional benefits in cosmetic and dermatological formulations or compositions, and enhance the aesthetic and natural appearance of biological surfaces, such as skin, lips, hair, and nails.

A yet further embodiment of the invention provides a mesoporous material composition with or without (empty/air) loaded pores, that has optical properties which enhance the aesthetic and natural appearance of a biological surface, while minimizing or eliminating the use of pigments or dyes for coverage, and yet resulting in optical blurring and diffuse light transmittance, light scattering and reflection, thereby reducing the appearance of dermatological signs of chronological aging, photo-aging, hormonal aging, and/or actinic aging; reducing the appearance of lines and/or wrinkles; reducing the noticeability of facial lines and wrinkles, facial wrinkles on the cheeks, forehead, perpendicular wrinkles between the eyes, horizontal wrinkles above the eyes, and around the mouth, marionette lines, and particularly deep wrinkles or creases; reducing the appearance and/or depth of lines and/or wrinkles; improving the appearance of suborbital lines and/or periorbital lines; reducing the appearance of crow's feet; improving the appearance of rejuvenating and/or revitalizing skin, decreasing the appearance of aging skin; reducing the appearance of skin fragility; reducing the appearance of a loss of glycosaminoglycans and/or collagen; reducing the appearance of estrogen imbalance; reducing the appearance of skin atrophy; reducing the appearance of hyperpigmentation; reducing the appearance of skin discoloration; improving the appearance of skin tone, radiance, clarity and/or tautness; reducing the appearance of sagging skin; improving the appearance of skin firmness, plumpness, suppleness and/or softness; improving the appearance of procollagen and/or collagen production; improving the appearance of skin texture and/or retexturization; improving the appearance of skin barrier repair and/or function; improving the appearance of skin contours; improving the appearance of decreased skin luster and/or brightness; improving the appearance of dermatological signs of fatigue and/or stress; improving the appearance of environmental stress; improving the appearance of cellular aging; improving the appearance of skin dehydration; improving the appearance of elastic and/or resilient skin; improving the appearance of microcirculation; decreasing the appearance of cellulite formation; or any combinations thereof.

Another embodiment of the invention relates to a method of improving the aesthetic or natural appearance of a biological surface comprising applying to the biological surface, including but not limited to, keratinous tissue, skin, hair and nails, the inventive composition comprising a mesoporous material having the characteristics and properties described herein, in an amount effective to improve the aesthetic or natural appearance of the biological surface. Without being bound by theory, skin thickness variations associated with aging decreases with loss of estrogen. Furthermore, glycosaminoglycans, procollagens, and precollagens (precursors of collagen) and collagen, all extracellular matrix components in the skin, production and presence are important for softening fine lines, wrinkles, dimpling, blemishes, sagging, resulting in the appearance of thicker healthier skin.

Contacting a keratinous surface with the inventive composition comprising a loaded or unloaded mesoporous material results in the formation of a film on the keratinous surface, where the film produces a blurring effect, improves the natural appearance of the keratinous surface. Keratinous surfaces may be mottled with folds, wrinkles, creases, dimples, sagging, and relaxed cutaneous tissue resulting from physiological aging, cutaneous senescence, photo-induced actinic aging, as elastic fibers become looser and fibroblasts become less active and/or as the appearance of keratinization and oxydative stress (free radicals) results in a diminished cutaneous vascular network.

The biological surface may be any surface to which cosmetics, personal care products, dermatological, and pharmaceutical compositions are typically applied, including but not limited to skin, lips, hair, nails, and the like. The composition that is applied to biological surfaces improves or enhances the aesthetic appearance of skin by camouflaging the natural aging process, discoloration, chronic and cumulative damage to biological surfaces, and imperfections on the surface.

Embodiments of the invention relate to the discovery that mesoporous materials with pores loaded or empty/unloaded in a composition or formulation camouflage fine lines, wrinkles, pores, sagging, or other conditions due to aging. Thus, by relying on the optical properties of the inventive composition enables one to camouflage the signs of aging, dermatological degeneration of biological surfaces, and imperfections of biological surfaces, thereby improving the aesthetic and natural appearance of skin.

One embodiment of the invention relates to methods of applying the mesoporous material composition that is loaded or unloaded (air) to an affected area of the skin. The composition is preferably applied topically at least once daily, preferably twice a day, and remains on the affected area of the skin, where the affected area of the skin includes, but is not limited to, the face, neck, legs and thighs, scalp, and overall body. Topical compositions preferably have the aforementioned mesoporous material, loaded or unloaded, which improves the cosmetic and/or aesthetic appearance of skin, particularly of aging and/or inflamed skin.

In another embodiment, the composition may have a mesoporous material in an amount from about 0.1% to about 50%, preferably from about 0.2% to about 20%, and more preferably from about 0.3% to about 10%, based on the total weight of the composition, where the composition is useful in improving the natural and aesthetic appearance of, for example, skin.

The mesoporous material composition is useful in improving the natural and aesthetic appearance of a biological surface, including skin, lips, hair, and nails, when applied, preferably topically in a daily manner to the biological surface. The composition of the invention may also include, in addition to a carrier or vehicle, non-limiting examples of active ingredients useful in reducing, diminishing, or camouflaging medical and/or cosmetic conditions associated with aging, inflammation, and degeneration of the biological surface. Such conditions, as used herein, commonly include, but are not limited to, dermatological aging (chronological aging, hormonal aging and/or actinic aging), dermatitis, skin and hair fragility, hirsutism, rosacea, skin blemishes, sensitive skin, hyperpigmentation or hypopigmentation, thinning skin, roughness, keratosis, skin atrophy, wrinkles, lines, hyperplasia, fibrosis, and any combinations thereof. The active components of the present invention may also be useful in enhancing the general health, vitality, condition, and aesthetic appearance of the skin.

In accordance with the invention, compositions comprising mesoporous materials having the desired properties, whether loaded with a compatible material or with air, may be useful in topically applied formulations, anti-oxidants, anti-inflammatories, sunscreens, cosmetics, including makeup, and formulations for reducing dermatological signs of aging, including wrinkles, fine lines, and sagging skin, and the like. Also in accordance with this invention, additional constituents in a composition, may be formulated in a variety of product forms. The compositions may be prepared in targeted delivery systems, e.g. creams, lotions, moisturizers, gels, toners, serums, sprays, foams, powders, and the like, particularly for topical application and administration.

The present invention further provides the compositions comprising the loaded and unloaded mesoporous material, preferably for topical administration or for targeted delivery without inducing significant irritation. The inventive compositions are suitable for all skin types, such as sensitive, normal, dry, or oily, preferably sensitive to dry skin, as well as mature skin. In particular embodiments, the compositions may be suitable for dry skin. The compositions are applied to the skin for a period of time sufficient to enhance the natural and aesthetic appearance of skin. The compositions may be applied topically once, twice, or more daily to biological surfaces, including but not limited to skin, lips, and hair. The daily application may be applied for a period of one week, two weeks, four weeks, or more. The preferred composition or formulation may be applied and left on the affected area once daily. The consumer could preferably apply the inventive composition at a frequency and duration in which to accomplish and reflect a flawless, natural look.

The topical compositions may be formulated into liposomes which may comprise other additives or substances, and/or which may be modified to more specifically reach or remain at a site following administration. The compositions of embodiments of the present invention yield improvements to the aesthetic appearance of skin by camouflaging or improving upon at least one of the previously described conditions, or combinations thereof.

As will be appreciated by the practitioner, cosmetic treatments comprising compositions containing mesoporous materials loaded with or without additional materials, such as (noble) metal nanocrystals of the invention may be carried out, for example, by topically applying the cosmetic composition as described herein according to the routine technique for administering such compositions. The topical cosmetic, dermatological, or pharmaceutical composition preferably is applied once daily for a period of at least one week, but may include a period of about 2, 4, 8, 12 or more weeks. The cosmetic composition is preferably applied to the face and neck, but may be applied to any area of skin in need of aesthetic improvement, where the cosmetic composition remains on the affected area of skin, and preferably not removed or rinsed off the skin. Routine and commonly practiced techniques encompass the application of creams, lotions, gels, sera, ointments, makeup, sunscreen compositions, or the like, to the skin. Preferably the cosmetic composition is a topical leave on formulation, where spraying as a form of application is also envisioned.

Another embodiment of the invention encompasses compositions comprising a cosmetically, dermatologically, or pharmaceutically acceptable formulation which is suitable for contact with living mammalian tissue, including human tissue, or synthetic equivalents thereof, with virtually no adverse physiological effect to the user. Compositions embraced by this invention can be provided in any cosmetically and/or dermatologically suitable form, preferably as a lotion or cream, but also in an anhydrous or aqueous base, as well as in a sprayable liquid form. Other suitable cosmetic product forms for the compositions of this invention include but are not limited to, for example, an emulsion, a cream, a balm, a gloss, a lotion, a foam, a mask, a serum, a toner, an ointment, a mousse, a patch, a pomade, a solution, a spray, a wax-based stick, or a towelette. In addition, the compositions contemplated by this invention can include one or more compatible cosmetically acceptable adjuvants commonly used and known by the skilled practitioner, such as fragrances, emollients, humectants, preservatives, vitamins, chelators, thickeners, perilla oil or perilla seed oil (WO 01/66067 to a “Method of Treating a Skin Condition,” incorporated herewith) and the like, as well as other botanicals such as aloe, chamomile, and the like, and as further described below. Pigments, dyes, and colorants would not be required or necessary, but could be used if so desired and if they do not interfere with the optical blurring and reflective properties of the composition. Preferably the mesoporous material in a cosmetic application would be used as an alternative to colorants, pigments, or dyes because the mesoporous material provides increased light diffusion properties comparable to light diffusing pigments, such as but not limited to nylon, PMMA, etc. while maintaining relatively high reflectance. However, mesoporous materials which provide color, such as, for example, mesoporous iron oxide, may be used to in combination with the mesoporous TiO₂ to match skin tone. Both of these mesoporous materials would have high reflectance and diffused transmittance properties.

The mesoporous materials, either loaded or unloaded, of the present invention may be contained in a cosmetically, dematologically, physiologically, and pharmaceutically acceptable vehicle, medium, diluent or carrier, for use in reducing, ameliorating, or preventing the dermatological signs associated with aging and inflammation of biological surfaces. In an embodiment embracing topical applications, the compositions of this invention comprise a medium (vehicle, diluent or carrier) that is compatible with mammalian biological surfaces, including skin, lips, hair and nails. The compositions can be formulated as an aqueous phase, an oil phase, alcohol, or aqueous/alcohol-based solutions, ointments, creams, lotions, gels, a wax-in-water emulsion, or water-in-oil, oil-in-water, of water-oil-water triple emulsions having the appearance of a cream or gel, microemulsions, or aerosols.

The aqueous phase is a mixture of one or more water soluble or water dispersible ingredient, which can be liquid, semi-solid or solid at room temperature (25° C.). The vehicle comprises or can be in the form of a suspension, dispersion or solution in water or an aqueous-alcoholic vehicle, which may contain a thickener or gellant. A person skilled in the art can select the appropriate cosmetic form, the ingredients contained therein, as well as the method for preparing it, on the basis of the knowledge that the skilled artisan possesses.

In one embodiment, the composition may include an aqueous phase which may contain water or a mixture of water and at least one hydrophilic organic solvent in particular an alcohol, especially a linear or branched lower monoalcohol containing from 2 to 5 carbon atoms, e.g., ethanol or propanol; a polyol, e.g., propylene glycol, sorbitol, glycerol, diglycerol, panthenol, or polyethylene glycol, and mixtures thereof. This aqueous phase may represent from about 0.5 weight % to about 99.99 weight %, based upon the total weight of the composition.

Yet another embodiment when the composition of the invention is in the form of an emulsion, the composition may also optionally comprise a surfactant, preferably in an amount of from about 0.1 weight % to about 30 weight %, and in particular, from about 1 weight % to about 20 weight %, based upon the total weight of the composition.

In a further embodiment of the invention, the composition may also comprise a thickening polymer such as an amphiphilic polyurethane, a polyacrylic homopolymer or copolymer, a polyester, or a hydrocarbon-based resin. Other non-limiting polymers include, homopolymers or copolymers of: vinyl esters of an aliphatic aid having 1 to 18 carbon atoms, such as vinyl acetate; acrylic acid esters and methacrylic acid esters of an alcohol having 1 to 18 carbon atoms, such as methyl acrylate, ethyl acrylate, butyl acrylate, 2-ethyhexyl acrylate, methyl methacrylate, ethyl methacrylate and butyl methacrylate; and mono and di-ethylenically unsaturated hydrocarbons, including ethylene iso-butylene, styrene and aliphatic dunes, including butadiene, isoprene and chloroprene.

One embodiment of the invention further relates to a composition of the invention which may also comprise an oil phase containing oil soluble or oil dispersible ingredients that are liquid at room temperature (25° C.) and/or oily or waxy substances that are solid at room temperature, such as waxes, semi-solids, gums, and mixtures thereof. This oily phase may also contain organic solvents.

Suitable oily materials that are liquid at room temperature, often referred to as oils, include hydrocarbon-based oils of animal origin such as perhydrosqualene; hydrocarbon-based plant oils such as liquid triglycerides of fatty acids of 4 to 10 carbon atoms, for instance, heptanoic or octanoic acid triglycerides, or oils such as sunflower oil, corn oil, soybean oil, grapeseed oil, castor oil, avocado oil, caprylic/capric acid triglycerides, jojoba oil; linear or branched hydrocarbons of mineral or synthetic origin such as liquid paraffins and derivatives thereof, petroleum jelly; synthetic esters and ethers, in particular esters of fatty alcohols, namely; for example, isopropyl myristate, 2-ethylhexyl palmitate, 2-octyldodecyl stearate, isostearyl isostearate; hydroxylated esters such as isostearyl lactate, octyl hydroxystearate, octyldodecyl hydroxystearate, heptanoates, octanoates and decanoates of fatty alcohols; polyol esters such as propylene glycol dioctanoate, neopentyl glycol diheptanoate, diethylene glycol diisononanoate, and pentaerythritol esters; fatty alcohols containing from 12 to 26 carbon atoms such as octyldodecanol, 2-butyloctanol, 2-hexyldecanol, 2-undecylpentadecanol, oleyl alcohol; partially hydrocarbon-based fluoro oils and/or fluorosilicone oils; silicone oils such as volatile or non-volatile, linear or cyclic polydimethylsiloxanes (PDMS) that are liquid or semisolid at room temperature such as cyclomethicones and dimethicones, optionally comprising a phenyl group, for instance phenyl trimethicones, siloxanes, and mixtures thereof. These oils are usually present in an amount of about 0 weight % to about 90 weight %, preferably from about 1 weight % to about 80 weight % by weight of the oil phase.

The oil phase of the composition of the invention may also comprise one or more cosmetically acceptable organic solvents. These solvents are present in an amount of about 0 weight % to about 60 weight %, preferably about 1 weight % to about 30 weight %, based on the total weight of the composition, and may be selected from the group consisting of lipophilic organic solvents, amphiphilic organic solvents and mixtures thereof. Suitable solvents which may be used in the composition of the invention include acetic acid esters such as methyl, ethyl, butyl, amyl or 2-methoxyethyl acetate; isopropyl acetate; hydrocarbons such as toluene, xylene, p-xylene, hexane or heptane; ethers containing at least 3 carbon atoms, and mixtures thereof.

The composition of the invention may further comprise any ingredient conventionally used in the cosmetics field. These ingredients include preserving agents, aqueous phase thickeners (polysaccharide biopolymers, synthetic polymers) and fatty-phase thickeners, fragrances, hydrophilic and lipophilic active agents, and mixtures thereof. The amounts of these various ingredients are those conventionally used in the cosmetics field to achieve their intended purpose, and range typically from about 0.01 weight % to about 20 weight %, based upon the total weight of the composition. The nature of these ingredients and their amounts must be compatible with the production of the compositions of the invention.

The composition of the invention may also comprise an additional particulate phase, typically present in an amount of about 0 weight % to about 30 weight %, based upon the total weight of the composition, preferably from about 0.05 weight % to about 20 weight %, and which can comprise pearlescent agents and/or fillers used in cosmetic compositions. Suitable pearlescent agents include mica coated with titanium oxide or with iron oxide.

Fillers are normally present in an amount of about 0 weight % to about 30 weight %, based on the total weight of the composition, preferably about 0.5 weight % to about 15 weight %. Suitable fillers include talc, silica, zinc stearate, mica, kaolin, nylon (in particular orgasol) powder, polyethylene powder, Teflon®, starch, boron nitride, copolymer microspheres such as Expancel® (Nobel Industrie; Sweden), Polytrap® (Dow Corning, Inc.; Midland, Mich.), and silicone resin microbeads (Tospearl®; GE Toshiba Silicones; Japan).

Although the composition of the invention is preferably utilized without colorants, pigments, or dyes as they may alter the desired optical properties, it is also contemplated that compositions may contain colorants, pigments, or dyes such that their presence which alters optical properties will be compensated in order to produce a desired blurring effect, enhanced diffused transmittance, and optimal reflection. Suitable inorganic pigments include, but are not limited to, titanium oxide, zirconium oxide and cerium oxide, as well as zinc oxide, iron oxide, chromium oxide and ferric blue. Suitable organic pigments include, but barium, strontium, calcium, and aluminium lakes and carbon black.

The oil phase of the compositions of the invention may comprise one or more waxes, gums, or mixtures thereof. The waxes include hydrocarbon-based waxes, fluoro waxes and/or silicone waxes and can be of plant, mineral, animal and/or synthetic origin. In particular, the waxes have a melting point of greater than 25° C., preferably greater than 45° C. The compositions of the present invention may contain from about 0 weight % to about 20 weight % waxes, based upon the total weight of the composition.

The gums are generally high molecular weight polydimethylsiloxanes (PDMSs), cellulose gums or polysaccharides, and the semi-solid materials are generally hydrocarbon-based compounds, such as, but not limited to, lanolins and derivatives thereof, or alternatively PDMSs. The compositions of the present invention may contain from about 0 weight % to about 20 weight % gums, based upon the total weight of the composition, typically from about 0.1 weight % to about 10 weight %.

More particularly, the compositions for topical application can be in the form of a protective care composition for the skin, preferably for the face, the neck, the hands, the feet, or other areas of the body. Non-limiting examples include day creams or lotions, night creams or lotions, moisturizer, salves, sunscreen creams, fluid lotions, oils, ointments, gels, body milks, makeup (a foundation, a bronzer), artificial tanning compositions, emulsifiers, or a solid which is poured or cast as a stick or a dish, for example. The mesoporous materials are ideal for use in a foundation product because it could achieve high camouflage and blurring effects to result in the perception of a natural appearance.

In another embodiment, the topical compositions of the present invention may also include one or more of the following: a skin penetration enhancer, an emollient, a skin plumper, an optical diffuser, a sunscreen, an exfoliation promoter, and an antioxidant. Details with respect to these and other suitable cosmetic ingredients can be found in the International Cosmetic Ingredient Dictionary and Handbook, 10th Edition (2004) (“INCI”), published by the Cosmetic, Toiletry, and Fragrance Association (CTFA), at pp. 2177-2299, which is herein incorporated by reference in its entirety.

An emollient provides the functional benefits of enhancing skin smoothness, reducing the appearance of fine lines and coarse wrinkles, and moisturizing. Non-limiting examples include isopropyl myristate, petrolatum, isopropyl lanolate, silicones (e.g., methicone, dimethicone), oils, mineral oils, fatty acid esters, or any mixtures thereof. The emollient is preferably present from about 0.1 weight % to about 50 weight % of the total weight of the composition.

A skin plumper serves as a collagen enhancer to the skin. An example of a suitable, and preferred, skin plumper is palmitoyl oligopeptide. Other skin plumpers are collagen and/or glycosaminoglycan (GAG) enhancing agents. The skin plumper is preferably present from about 0.1 weight % to about 20 weight % of the total weight of the composition.

In addition to the mesoporous optical diffuser, additional optical diffusers or soft focus materials that change the surface optical properties of skin, resulting in a visual blurring and softening of, for example, lines and wrinkles are contemplated. Examples of optical diffusers that can be used in the present invention include, but are not limited to, boron nitride, mica, nylon, polymethylmethacrylate (PMMA), polyurethane powder, sericite, silica, silicone powder, talc, Teflon, titanium dioxide, zinc oxide, or any mixtures thereof. The optical diffuser is preferably present from about 0.01 weight % to about 20 weight % of the total weight of the composition.

A sunscreen protects the skin from damaging ultraviolet rays. In an illustrative embodiment of the invention, the sunscreen would provide both UVA and UVB protection, by using either a single sunscreen or a combination of sunscreens. Among the sunscreens that can be employed in the present compositions are avobenzone, cinnamic acid derivatives (such as octylmethoxy cinnamate), octyl salicylate, oxybenzone, non-mesoporous titanium dioxide, zinc oxide, or any mixtures thereof. The sunscreen may be present from about 1 weight % to about 30 weight % of the total weight of the composition. The addition of a sunscreen may protect the skin from ultraviolet radiation.

The compositions of the invention having sunscreen bring about additional improvements to the aesthetic appearance of skin, including at least one of the following: minimizes sunburning, minimizes tanning, and reduces redness.

In an embodiment of the invention, compositions may also have one or more exfoliation promoters. Suitable examples of an exfoliation promoter that can be used in the present compositions include alpha hydroxy acids (AHA); benzoyl peroxide; beta hydroxy acids; keto acids, such as pyruvic acid, 2-oxopropanoic acid, 2-oxobutanoic acid, and 2-oxopentanoic acid; oxa acids as disclosed in U.S. Pat. Nos. 5,847,003 and 5,834,513 (the disclosures of which are incorporated herein by reference); salicylic acid; urea; or any mixtures thereof. The preferred exfoliation promoters are 3,6,9-trioxaundecanedioic acid, glycolic acid, lactic acid, or any mixtures thereof. (See also, INCI at p. 2205).

When an embodiment of the invention includes an exfoliation promoter, the composition has about 0.1 weight % to 30 weight %, preferably about 1 weight % to about 15 weight % and more preferably about 1 wt % to about 10 wt %, of the exfoliation promoter based on the total weight of the composition.

An antioxidant functions, among other things, to scavenge free radicals from skin to protect the skin from environmental aggressors. Examples of antioxidants that may be used in the present compositions include compounds having phenolic hydroxy functions, such as ascorbic acid and its derivatives/esters; beta-carotene; catechins; curcumin; ferulic acid derivatives (e.g. ethyl ferulate, sodium ferulate); gallic acid derivatives (e.g. propyl gallate); lycopene; reductic acid; rosmarinic acid; tannic acid; tetrahydrocurcumin; tocopherol and its derivatives; uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. Compositions of the present invention may have an antioxidant preferably from about 0.001 wt % to about 10 wt %, and more preferably from about 0.01 wt % to about 5 wt %, of the total weight of the composition. (See also, INCI at p. 2184).

In an embodiment of the invention, the composition may also have one or more of the following cosmetic and pharmaceutical active agents, excipients, ingredients, or adjuvants: anesthetics, antibiotics, e.g., erythromycins and tetracyclines, salicylic acids, anti-allergenics, antifungals, antiseptics, anti-irritants, anti-inflammatory agents, antimicrobials, analgesics, nitric oxide synthase inhibitors, insect repellents, self-tanning agents, skin penetration enhancers, skin cooling agents, chelating agents, colorants including dyes, lakes and pigments that may be untreated or chemically surface treated to improve wettability or some other property, demulcents, emollients, emulsifiers, fragrances, humectants, lubricants, skin protectants, moisturizers, pH adjusters, preservatives, stabilizers, surfactants, thickeners, film formers, plasticizers, viscosity modifiers, vitamins, or any mixtures thereof. The amounts of these various substances are those that are conventionally used in the cosmetic or pharmaceutical fields to achieve their intended purposes, for example, they may constitute from about 0.01% to 20% of the total weight of the composition.

Non-limiting examples of active agents for formulating into the compositions of the invention include those reagents having an effect on the treatment of wrinkles and/or fine lines, in addition to the actives as described, such as keratolytic agents, i.e., an active agent having desquamating, exfoliant, or scrubbing properties, or an active agent which can soften the horny layer of the skin. Other examples of anti-wrinkle or anti-fine line active agents include hydroxy acids and retinoids. These agents can be formulated, for example, in amounts of from about 0.0001% to 5% by weight relative to the total weight of the composition.

Suitable hydroxy acids include, for example, glycolic acid, lactic acid, malic acid, tartaric acid, citric acid, 2-hydroxyalkanoic acid, mandelic acid, salicylic acid and alkyl derivatives thereof, including 5-n-octanoylsalicylic acid, 5-n-dodecanoylsalicylic acid, 5-n-decanoylsalicylic acid, 5-n-octylsalicylic acid, 5-n-heptyloxysalicylic acid, 4-n-heptyloxysalicylic acid and 2-hydroxy-3-methylbenzoic acid or alkoxy derivatives thereof, such as 2-hydroxy-3-methyoxybenzoic acid.

Emulsifiers are typically present in the compositions of the invention in an amount of about 0.01 weight % to 30 weight %, by weight and preferably from about 0.1 weight % to 30 weight % by weight relative to the total weight of the composition. However, not all compositions will necessarily include emulsifiers. (See, e.g., INCI at p. 2276-2285).

Non-limiting examples of suitable thickening agents include xanthan gum, hydroxypropyl cellulose, hydroxyethyl cellulose, carbomer, gum acacia, Sepigel 305 (available from Seppic Co., France), and clays such as magnesium aluminum silicate. (See, e.g., INCI at p. 2293-2299).

The topical compositions of the present invention may include, and their utility can be enhanced by one or more humectants, such as ureas, pyrrolidone carboxylic acids, amino acids, sodium hyaluronates, certain polyols and other compounds with hygroscopic properties. (See INCI at p. 2244).

The general activity and mildness to skin of the present topical compositions can also be enhanced by neutralization to pH about 3.5 to about 7.0, most preferably from pH about 3.7 to about 5.6. This neutralization is preferably accomplished with one or more of ammonium hydroxide, potassium hydroxide, sodium hydroxide, arginine or other amino acids, and/or triethanolamine.

Exemplary retinoids include, without limitation, retinoic acid (e.g., all-trans or 13-cis) and derivatives thereof, retinol (Vitamin A) and esters thereof, such as retinol palmitate, retinol acetate and retinol propionate, and salts thereof.

The mesoporous materials, of the present invention may be contained in a cosmetically or dematologically acceptable vehicle, medium, diluent or carrier. The topical composition comprising the loaded or unloaded mesoporous materials may be further formulated according to procedures known in the art to provide cosmetic compositions such as emulsions, gels, creams, lotions, masks, toners, serums, oils, water-in-oil, oil-in-water, water-oil-water triple emulsions having the appearance of a cream or gel, microemulsions, ointments, pastes, sticks, cakes, pencils, aerosol, and essences, as well as other topical cosmetic vehicles. It is also contemplated that topical compositions of the present invention can be incorporated into delivery systems such as liposomes and topical patches, tapes, and sprays.

In addition, the compositions may be in the form of vesicular dispersions containing ionic and/or nonionic lipids, as described above. Dosage units suitable for such compositions are formulated according to the conventional knowledge and techniques used in the art.

In a further embodiment of the invention, the compositions for topical application may be in the form of a protective care composition for the skin, preferably for the face, the neck, the hands, the feet, or other areas of the body. Non-limiting examples include day creams or lotions, night creams or lotions, sunscreen creams, lotions, or oils, body milks, makeup (a foundation), artificial tanning compositions, depilatories, and patches.

### EXAMPLES

The following examples describe specific aspects of the invention to illustrate the invention and provide a description of the present methods for those skilled in the art. The examples should not be construed as limiting the invention, as the examples merely provide specific methodology useful in the understanding and practice of the invention and its various aspects.

### Example 1

Diffused transmittance is typically used to quantify soft focus or blurring efficiency. The Color Eye 7000 Spectrophotometer (GretagMagbeth; New Windsor, N.Y.) was used for measuring diffused transmittance. The color eye spectrophotometer allows measurement of films in three different modes: total transmittance, direct transmittance, and reflectance. Diffused transmittance can be obtained by subtracting direct transmittance from total transmittance. Efficiency of blurring or soft focus effects or the ability to hide lines and wrinkles is believed to be a function of diffused transmittance parameters (“Quantification of the Soft Focus Effect.” Cosmetics & Toiletries; Vol 111, July 1996; pp 57).

Physical blends were prepared using a speed mixer with silicone acrylate copolymer (KP 550 from Shin Etsu), which is 40% active (polymer) in isododecane with either pigment grade TiO₂ or mesoporous TiO₂. Solutions of each were cast on clean (optically transparent/clear) glass plates and dried overnight to form dry films with a resulting dry film thickness of 10 microns.

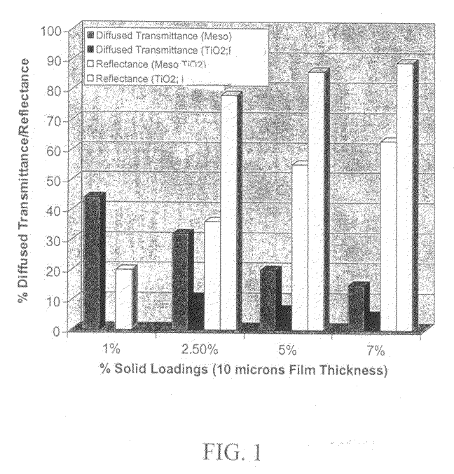

Total transmittance, direct transmittance and reflectance were measured on each sample using the color eye spectrometer. Reflectance or transmittance was obtained by averaging light intensity between a wavelength of 450 to 700 nm. Each film was measured at three different locations and each measurement was an average of 3 repeat measurements. FIG. 1 shows diffused transmittance and reflectance of a 10 micron film of mesoporous TiO₂ compared to a film of pigment grade TiO₂ at various loadings (wt %). Each film was measured at three different locations and each measurement was an average of 3 repeated measurements.

High diffused transmittance is an indication of high soft focus effect or blurring effect, while reflectance is a measure of coverage. When comparing the Pigment Grade TiO₂ with Mesoporous TiO₂, the mesoporous material demonstrated a higher percent of enhanced diffused transmittance with reasonably high reflectance at various solid loading concentrations or weight percent when compared to pigment grade TiO₂. The pigment grade TiO₂ has high reflectance but low diffused transmittance creating a mask-like appearance due to its high reflectance and high coverage properties. The mesoporous TiO₂ is advantageous because it has a lower reflectance and higher diffused transmittance, thereby providing optimal coverage and blurring effect. Thus, it is evident from the data that mesoporous TiO₂ would have higher blurring effect of lines, wrinkles, and skin imperfections, while maintaining good coverage when used in a topical cosmetic, for example, a foundation.

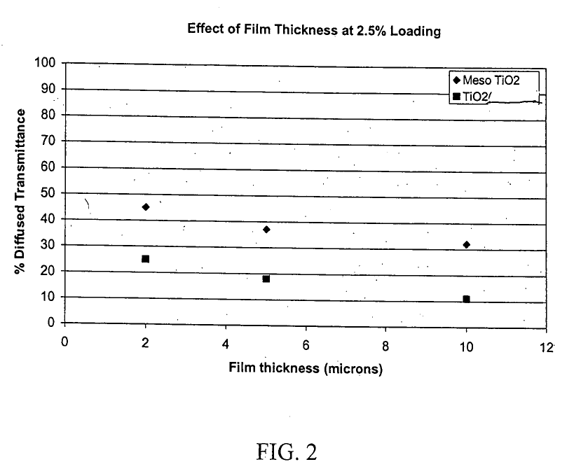

Using mesoporous materials in a cosmetic would provide a good balance between blurring and coverage. FIG. 2 demonstrates film thickness effects on diffused transmittance of films containing mesoporous TiO₂ and pigment grade TiO₂. At a solid load of 2.5%, mesoporous TiO₂ had a percent diffused transmittance about 2-3 fold higher than pigment grade TiO₂ at every film thickness tested. The data therefore support that mesoporous TiO₂ has a more efficient blurring property compared to pigment grade TiO₂.

Another advantage of using mesoporous metal oxide such as TiO₂ is that a reasonably high reflectance can be obtained compared to other traditional soft focus materials such as PE (polyethylene), silica, PMMA (Polymethyl-methacrylate), etc. In Table 1 below, the reflectance of TiO₂ compared to other traditional soft focus materials was found to be much higher. The various materials were mixed with silicone acrylate and measured as described above.

TABLE 1

| | AVG. | DIFFUSED |
|---|---|---|
| | REFLECTANCE | TRANSMITTANCE |
| IN SILICONE ACRYLATE | (Std Deviation) | (Std Deviation) |
| 5% Nylon | 12 (0) | 67 (0) |
| 5% PMMA | 13 (0) | 72 (0) |
| 5% Silica | 13 (1) | 27 (1) |
| 5% polyethylene | 12 (0) | 70 (2) |
| 5% Mesoporous TiO₂ | 53 (1) | 20 (1) |
| 5% Pigment Grade TiO2 | 80 (1) | 10 (1) |

### Example 2

Any composition comprising ingredients normally used in formulating foundations may be used. However, one of the advantages is that mesoporous TiO₂ may be used as an alternative to pigment or in conjunction with pigments if so desired. In Table 2 below, examples of foundation formulations are prepared with and without pigments.

TABLE 2

| INGREDIENTS | I | II | III | IV | V | VI | VII | VIII | IX | X |
|---|---|---|---|---|---|---|---|---|---|---|
| Mesoporous Metal Oxide | 2 | 4 | 5 | 6 | 7 | 8 | 7 | 5 | 10 | 7 |
| Pigments | 2 | 3 | 2 | 3 | 2 | 3 | 2 | 4 | 0 | 0 |
| Fillers | 5 | 4 | 5 | 4 | 3 | 3 | 3 | 3 | 3 | 0 |
| Silicone Fluid | 3 | 2 | 10 | 5 | 2 | 3 | 12 | 14 | 15 | 10 |
| Elastomer | 10 | 15 | 12 | 14 | 14 | 10 | 25 | 25 | 20 | 14 |
| Optical Diffuser | 0 | 0 | 2 | 3 | 5 | 3 | 5 | 5 | 9 | 5 |
| Emulsifier | 10 | 10 | 10 | 10 | 10 | 10 | 0 | 0 | 0 | 10 |
| Isododecane | 11.8 | 10 | 4.8 | 1.8 | 2.8 | 3 | 18 | 17 | 17 | 2.8 |
| Silicone Gum | 1 | 1 | 2 | 0 | 0 | 0 | 2 | 3 | 3 | 0 |
| Silicone Solvent | 10 | 10 | 10 | 10 | 11 | 15 | 26 | 24 | 23 | 11 |
| Thickener | 4 | 2 | 4 | 4 | 3 | 4 | 0 | 0 | 0 | 3 |
| Water | 41 | 38.8 | 33 | 39 | 40 | 37.8 | 0 | 0 | 0 | 37 |
| NaCl | 0.2 | 0.2 | 0.2 | 0.2 | 0.2 | 0.2 | 0 | 0 | 0 | 0.2 |
| Total % | 100 | 100 | 100 | 100 | 100 | 100 | 100 | 100 | 100 | 100 |

The content of all patents, patent applications, published articles, abstracts, books, reference manuals and abstracts, as cited herein are hereby incorporated by reference in their entireties to more fully describe the state of the art to which the invention pertains. All concentrations recited in the specification and claims are reported as weight percents, unless otherwise indicated.

It should be understood that the foregoing description is only illustrative of the present invention. Various alternatives and modifications can be devised by those skilled in the art without departing from the invention. Accordingly, the present invention is intended to embrace all such alternatives, modifications and variations that fall within the scope of the appended claims.

What is claimed is:

1. A topical composition comprising:
(a) an effective amount of a mesoporous material sufficient to alter the appearance of a biological surface; and
(b) an acceptable vehicle.

2. The composition of claim 1, wherein the mesoporous material comprises a metal oxide, combinations thereof, or a hybrid of the metal oxide and a polymer.

3. The composition of claim 2, wherein the metal oxide comprises titanium oxide, aluminum oxide, silicon dioxide, zinc oxide, or iron oxide.

4. The composition of claim 2, wherein the hybrid is polysilsesquioxane.

5. The composition of claim 1, wherein the mesoporous material is present in an amount from about 0.1 weight % to about 50 weight %, based upon the total weight of the composition.

6. The composition of claim 1, wherein the mesoporous material is present in an amount from about 0.2 weight % to about 20 weight %, based upon the total weight of the composition.

7. The composition of claim 1, wherein the mesoporous material has a pore volume of about 0.05 cm³/g to about 2 cm³/g.

8. The composition of claim 1, wherein the mesoporous material has a pore size of about 2 nanometers to about 50 nanometers.

9. The composition of claim 1, wherein the mesoporous material has a pore size less than the wavelength of visible light.

10. The composition of claim 1, wherein the mesoporous material has a refractive index from about 1.4 to about 3.5.

11. The composition of claim 1, wherein the mesoporous material has a surface area in the range of 20 m²/g to 700 m²/g.

12. The composition of claim 1, wherein a film of the mesoporous material has a reflectance of less than about 55% and a diffused transmittance of greater than about 18%, and wherein said film comprises 5% mesoporous material and a thickness of 10 microns.

13. The composition of claim 1, wherein the mesoporous material has an ordered or disordered distribution of pores.

14. The composition of claim 13, wherein the pores are loaded with metal nanocrystals.

15. The composition of claim 14, wherein the metal nanocrystals comprise gold, silver, copper, platinum, palladium, tantalum, or iridium.

16. The composition of claim 13, wherein the pores are loaded with a fluorescent material.

17. The composition of claim 1, wherein the mesoporous material is present in an amount effective to improve the aesthetic and natural appearance of the biological surface.

18. The composition of claim 1, wherein the mesoporous material absorbs sebum.

19. The composition of claim 1, wherein the composition is applied to the biological surface to optically improve the aesthetic appearance of biological surface.

20. The composition of claim 19, wherein the improvement in aesthetic appearance comprises: reducing the appearance of dermatological signs of chronological aging, photo-aging, hormonal aging, and/or actinic aging; reducing the appearance of lines and/or wrinkles; reducing the noticeability of facial lines and wrinkles, facial wrinkles on the cheeks, forehead, perpendicular wrinkles between the eyes, horizontal wrinkles above the eyes, and around the mouth, marionette lines, and particularly deep wrinkles or creases; reducing the appearance and/or depth of lines and/or wrinkles; improving the appearance of suborbital lines and/or periorbital lines; reducing the appearance of crow's feet; improving the appearance of rejuvenating and/or revitalizing skin, decreasing the appearance of aging skin; reducing the appearance of skin fragility; reducing the appearance of a loss of glycosaminoglycans and/or collagen; reducing the appearance of estrogen imbalance; reducing the appearance of skin atrophy; reducing the appearance of hyperpigmentation; reducing the appearance of skin discoloration; improving the appearance of skin tone, radiance, clarity and/or tautness; reducing the appearance of sagging skin; improving the appearance of skin firmness, plumpness, suppleness and/or softness; improving the appearance of procollagen and/or collagen production; improving the appearance of skin texture and/or retexturization; improving the appearance of skin barrier repair and/or function; improving the appearance of skin contours; improving the appearance of decreased skin luster and/or brightness; improving the appearance of dermatological signs of fatigue and/or stress; improving the appearance of environmental stress; improving the appearance of cellular aging; improving the appearance of skin dehydration; improving the appearance of elastic and/or resilient skin; improving the appearance of microcirculation; decreasing the appearance of cellulite formation; or any combinations thereof.

21. A method of improving the aesthetic or natural appearance of a biological surface comprising, applying to the biological surface the topical composition of claim 1, in an amount effective to improve the aesthetic or natural appearance of the biological surface.

22. The method of claim 21, wherein the improvement in aesthetic appearance comprises: reducing the appearance of dermatological signs of chronological aging, photo-aging, hormonal aging, and/or actinic aging; reducing the appearance of lines and/or wrinkles; reducing the noticeability of facial lines and wrinkles, facial wrinkles on the cheeks, forehead, perpendicular wrinkles between the eyes, horizontal wrinkles above the eyes, and around the mouth, marionette lines, and particularly deep wrinkles or creases; reducing the appearance and/or depth of lines and/or wrinkles; improving the appearance of suborbital lines and/or periorbital lines; reducing the appearance of crow's feet; improving the appearance of rejuvenating and/or revitalizing skin, decreasing the appearance of aging skin; reducing the appearance of skin fragility; reducing the appearance of a loss of glycosaminoglycans and/or collagen; reducing the appearance of estrogen imbalance; reducing the appearance of skin atrophy; reducing the appearance of hyperpigmentation; reducing the appearance of skin discoloration; improving the appearance of skin tone, radiance, clarity and/or tautness; reducing the appearance of sagging skin; improving the appearance of skin firmness, plumpness, suppleness and/or softness; improving the appearance of procollagen and/or collagen production; improving the appearance of skin texture and/or retexturization; improving the appearance of skin barrier repair and/or function; improving the appearance of skin contours; improving the appearance of decreased skin luster and/or brightness; improving the appearance of dermatological signs of fatigue and/or stress; improving the appearance of environmental stress; improving the appearance of cellular aging; improving the appearance of skin dehydration; improving the appearance of elastic and/or resilient skin; improving the appearance of microcirculation; decreasing the appearance of cellulite formation; or any combinations thereof.

23. The method of claim 21, wherein the mesoporous material comprises a metal oxide, combinations thereof, or a hybrid of the metal oxide and polymers.

24. The method of claim 23, wherein the metal oxide comprises titanium oxide, aluminum oxide, zinc oxide, silicon dioxide, or iron oxide.

25. The method of claim 21, wherein the mesoporous material is present in an amount from about 0.1 weight % to about 50 weight %, based upon the total weight of the composition.

26. The method of claim 21, wherein the mesoporous material is present in an amount from about 0.2 weight % to about 20 weight %, based upon the total weight of the composition.

27. The method of claim 21, wherein the mesoporous material has a pore volume of about 0.05 cm³/g to about 2 cm³/g.

28. The method of claim 21, wherein the mesoporous material has a pore size of about 2 nanometers to about 50 nanometers.

29. The method of claim 21, wherein the mesoporous material has a pore size less than the wavelength of visible light.

30. The method of claim 21, wherein the mesoporous material has a refractive index from about 1.4 to about 3.5.

31. The method of claim 21, wherein the mesoporous material has a surface area is in the range of 20 m²/g to 700 m²/g.

32. The method of claim 21, wherein a film of the mesoporous material has a reflectance of less than about 55% and a diffused transmittance of greater than about 18%, and wherein said film comprises 5% mesoporous material and a thickness of 10 microns.

33. The method of claim 21, wherein the mesoporous material has an ordered or disordered distribution of pores.

34. The method of claim 33, wherein the pores are loaded with metal nanocrystals.

35. The method of claim 34, wherein the metal nanocrystals comprise precious metals, gold, silver, copper, platinum, palladium, tantalum, or iridium.

36. The method of claim 33, wherein the pores are loaded with a fluorescent material.

37. The method of claim 21, wherein the mesoporous material absorbs sebum.

38. A method for forming a film on a keratinous surface comprising: contacting said surface with the composition of claim 1, spreading the composition on the surface, and forming a film on the keratinous surface, wherein the film produces a blurring effect, improves the natural appearance of the keratinous surface, diffuses transmittance, reflects light, and/or is opaque.
